(19) United States
(12) Patent Application Publication
(10) Pub. No.: US 2020/0164162 A1
(43) Pub. Date: May 28, 2020

(54) MICRO-VAPORIZER WITH MULTIPLE LIQUID RESERVOIRS

(71) Applicant: Blackship Technologies Development LLC, North Chesterfield, VA (US)

(72) Inventors: Donovan Phillips, Richmond, VA (US); Yongjie Xu, Richmond, VA (US); Russell Paul Rogers, Richmond, VA (US)

(21) Appl. No.: 16/695,599

(22) Filed: Nov. 26, 2019

Related U.S. Application Data
(60) Provisional application No. 62/771,714, filed on Nov. 27, 2018.

(51) Int. Cl. A61M 11/04; A61M 15/06

(52) U.S. Cl. CPC A61M 11/042; A61M 2205/3334; A61M 2205/8206; A61M 15/06

(57) ABSTRACT

A personal vaporizer has a vaporizer case defining a vaporizer interior, a vaporization chamber within the vaporizer interior, an air flow passage from one or more air intake openings in the case to the vaporization chamber, a vaporization mixture flow passage extending from the vaporization chamber to an exit port, and a heating arrangement having at least one heating element disposed within the vaporization chamber. The heating arrangement is connected to a power source for selective activation by a user. A first fluid reservoir is configured for retaining a first vaporizable fluid within a first reservoir interior and a second fluid reservoir is configured for retaining a second vaporizable fluid within a second reservoir interior. A fluid transport arrangement is configured for transporting the first and second vaporizable fluids from the first and second fluid reservoirs into the vaporization chamber.

This application claims priority to U.S. Provisional Patent Application 62/771,714, filed Nov. 27, 2018, the complete disclosure of which is incorporated herein by reference.

### BACKGROUND OF THE INVENTION

The invention relates generally to micro-vaporizers and, more particularly, to micro-vaporizers having multiple reservoirs for separate storage of vaporizable fluids.

Micro-vaporizers are devices in which a vaporizable fluid is drawn from a storage reservoir into a chamber where it is heated to vaporization temperature by a heating element. The vaporized fluid is then drawn or forced from the chamber. In products such as electronic cigarettes (also known as e-cigarettes or personal vaporizers), the vaporized fluid is drawn from the chamber through a mouthpiece and inhaled by the user. In other products, the vaporized fluid is dispersed into the atmosphere.

The usual purpose of a device that uses a micro-vaporizer is to dispense one or more active substances using the vaporized fluid. In atmospheric dispensers, these substances may include materials such as deodorizing agents, fragrance, and insect repellant. In the case of personal vaporizers, the active substances typically include a flavorant (i.e., a flavoring agent or material) and nicotine. The flavorant and nicotine levels may be selected so as to mimic the experience of smoking a cigarette.

### SUMMARY OF THE INVENTION

An illustrative aspect of the invention provides a personal vaporizer comprising a vaporizer case defining a vaporizer interior. The personal vaporizer further comprises a vaporization chamber within the vaporizer interior, an air flow passage from one or more air intake openings in the case to the vaporization chamber, a vaporization mixture flow passage extending from the vaporization chamber to an exit port, and a heating arrangement comprising at least one heating element disposed within the vaporization chamber. The heating arrangement is connected to a power source for selective activation by a user. The personal vaporizer still further comprises a first fluid reservoir configured for retaining a first vaporizable fluid within a first reservoir interior, a second fluid reservoir configured for retaining a second vaporizable fluid within a second reservoir interior, and a fluid transport arrangement configured for transporting the first and second vaporizable fluids from the first and second fluid reservoirs into the vaporization chamber.

### BRIEF DESCRIPTION OF THE DRAWINGS

The invention can be more fully understood by reading the following detailed description together with the accompanying drawing, in which like reference indicators are used to designate like elements, and in which:

FIG. 1 is a section view of a personal vaporizer according to an embodiment of the invention;

FIG. 2 is a section view of a variation of the personal vaporizer of FIG. 1;

FIG. 3 is a section view of a personal vaporizer according to an embodiment of the invention;

FIG. 4 is a cross-sectional view of a portion of the personal vaporizer of FIG. 3;

FIG. 5 is a section view of a personal vaporizer according to an embodiment of the invention;

FIG. 6 is a section view of a variation of the personal vaporizer of FIG. 4;

FIG. 7 is a section view of a personal vaporizer according to an embodiment of the invention;

FIG. 8 is a section view of a personal vaporizer according to an embodiment of the invention; and

FIG. 9 is a section view of a portion of a personal vaporizer according to an embodiment of the invention.

### DETAILED DESCRIPTION OF THE INVENTION

One aspect of prior art vaporizers is that they use a single vaporizable fluid with a single set of characteristics. In some applications, however, it may be beneficial to combine the characteristics provided by one vaporizable fluid with the characteristics provided by another. In other applications, it may be desirable to separately store liquids that could otherwise be combined into a single vaporizable liquid. In either of these scenarios, it is advantageous to provide separate vaporizable fluid reservoirs within a single micro-vaporizer. It may also be advantageous to maintain two liquids in separate reservoirs, then heat them separately using different heating elements, each with temperature or other heating characteristics tailored to its particular liquid.

The present invention provides micro-vaporizers that use a plurality of vaporizable fluids, each stored in a separate reservoir. In some embodiments, the multiple fluids are separately drawn from their respective reservoirs and heated to vaporization, whereupon their respective vapor products are mixed with air and one another to form a single air/vapor mixture. Vaporization in such embodiments may be accomplished using single or multiple heat sources. In other embodiments, the multiple liquids, when drawn from their respective reservoirs, may be mingled or even thoroughly mixed with one another prior to vaporization.

In each of various embodiments of the invention, a micro-vaporizer comprises multiple vaporizable fluid sources from which vaporizable fluids, typically comprising one or more active materials, are drawn to or is otherwise presented to one or more heat sources that cause the fluids to be vaporized. The resulting vapor is mixed with air in a vaporization chamber, then passed to an exit chamber where it exits the device. In typical personal vaporizers, the exit chamber is defined by a mouthpiece (sometimes referred to as a “tip” or “drip tip”) and the combined air/vapor mixture is drawn through and out of the device by inhalation of a user.

As used herein, the term “active material” refers to any material that controllably alters or adds to the vaporization products of the device. Depending on the application, active materials can include, without limitation, plant material, minerals, deodorizing agents, fragrances, insect repellants, medications, and disinfectants and any material or structure containing or incorporating any of the foregoing.

In the specific instance of personal vaporizers, active materials may include flavorant substances that augment the flavorant of the vaporizable fluid. These may include, without limitation, marijuana, hemp, cannabidiol (cbd), citronella, geraniol, mint, thyme, tobacco, Salvia dorrii, Salvia, Passiflora incarnata, Arctostaphylos uva-ursi, Lobelia inflata, lemon grass, cedar wood, clove, cinnamon, coumarin, helio, vanilla, menthol, eucalyptus, peppermint, rosemary, lavender, licorice, and cocoa and any material or structure containing or incorporating any of the foregoing. The aforementioned active materials can be provided in varying concentrations or potency levels.

The invention will be described in more detail using examples and embodiments geared primarily to personal vaporizers. It will be understood, however, that the methods of the invention are not limited to such applications and can be applied to any micro-vaporizer device.

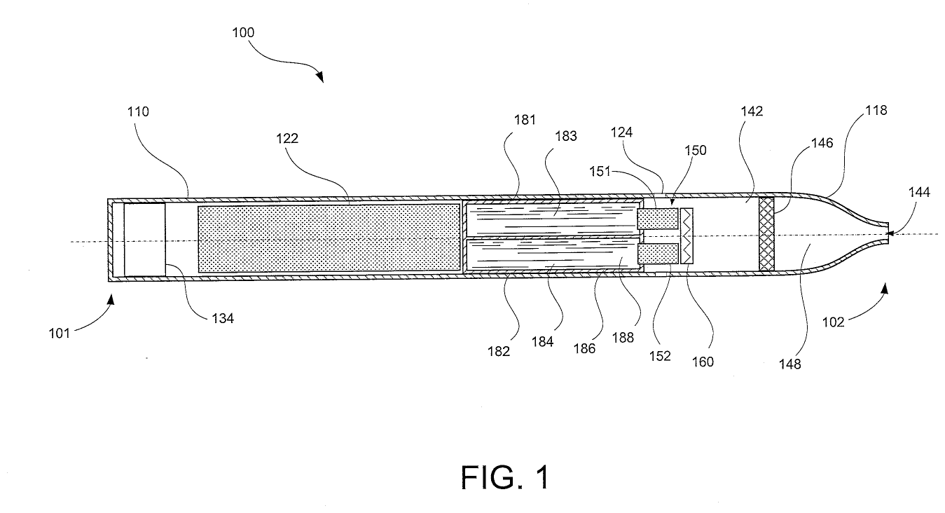

With reference to FIG. 1, a personal vaporizer 100 comprises a cylindrical casing 110 having a distal end 101 and a proximal end 102. At its proximal end 102, the casing 110 is formed into a mouthpiece 118 having a passage 144 providing fluid communication between the atmosphere and an exit chamber 148 inside the casing 110. The casing 110 also has one or more air holes 124 to allow air to flow from the atmosphere into a vaporization chamber 142 inside the casing 110 when a relative vacuum is applied at the mouthpiece passage 144 (e.g., by inhalation of a device user). The air drawn in through the air hole(s) 124 passes through a filter 146 which divides the vaporization chamber 142 and the exit chamber 148.

The personal vaporizer 100 further comprises two fluid reservoirs 181, 182 in which are disposed respective vaporizable fluids 183, 184. The fluid reservoirs 181, 182 may be configured as simple semicylinder tanks in which the fluids 183, 184 are disposed. In some embodiments, either or both of the reservoirs 181, 182 may be or include a housed or unhoused adsorptive or absorptive material or structure that retains the vaporizable fluid 183, 184. The vaporizer 100 also includes a fluid transport arrangement 150 that is configured for drawing the fluids 183, 184 into the vaporization chamber 142. The fluid transport arrangement 150 includes a first fluid transport structure 151 configured and positioned to be in contact with the vaporizable fluid 183 in the first reservoir 181 and for drawing the fluid 183 out of the reservoir 181 into the vaporization chamber 142 and into close proximity or in contact with a heating element 160. The fluid transport arrangement 150 also includes a second fluid transport structure 152 configured and positioned to be in contact with the vaporizable fluid 184 in the second reservoir 182 and for drawing the fluid 184 out of the reservoir 182 into the vaporization chamber 142 and into close proximity or in contact with a heating element 160. The heating element 160 may be configured to heat the vaporizable fluids 183, 184 through any conductive, convective, and/or radiative heat transfer mechanism. In typical vaporizers, the heating element 160 is or includes a resistance element in the form of a wire coil. In some cases, the resistance element is housed within a heat conductive casing.

The fluid transport structures 151, 152 of the personal vaporizer 100 may each be or comprise a wick or collection of wicking material. Typical personal vaporizer wicks are formed from organic fiber materials such as cotton, jute, flax, cellulose, or hemp. Some non-organic materials such as silica, carbon, and non-organic polymer fibers, ceramics and steel mesh may also be used. In general, vaporizer wicks can be formed from any material that is thermally stable and that provides sufficient wicking action to transport a particular vaporizable fluid from its reservoir to the heating element 184. It will be understood that the fluid transport structures 151, 152 can have different wicking characteristics. They can, for example, be formed from different materials and/or formed with different flow characteristics or other properties. In particular, the fluid transport structures 151, 152 may be tailored to the flow properties of their respective vaporizable liquids 183, 184.

The illustrative personal vaporizer 100 also comprises a battery 122 for powering the heating element 160 and a control unit 134. It will be understood that the configuration and relative positioning of the components of the personal vaporizer 100 may be widely varying and that additional components (e.g., an airflow controller for regulation of the amount of air flow through the holes 124) may be included.

To use the personal vaporizer 100, a user activates the first and second heating elements 160, 170 and draws air through the device by inhaling through the mouthpiece 118. The vaporizable fluids 183, 184 in the proximal end portions of the fluid transport structures 151, 152 are heated to their vaporization points by the heating element 160. Vapor from the vaporized fluids 183, 184 mixes with air drawn through the air holes 124 and the resulting air/vapor mixture is drawn through the filter 146 and the exit chamber 148 and out through the mouthpiece passage 144.

It will be understood that the personal vaporizer 100 is not limited to two fluid reservoirs/vaporizable fluids. The reservoir section of the personal vaporizer 100 could, for example, be divided into three (or more) reservoirs instead of the two semi-cylindrical reservoirs 181, 182 of FIG. 1. In such an embodiment, each reservoir may contain a different vaporizable fluid and the fluid transport arrangement 150 may include a fluid transport structure for each reservoir.

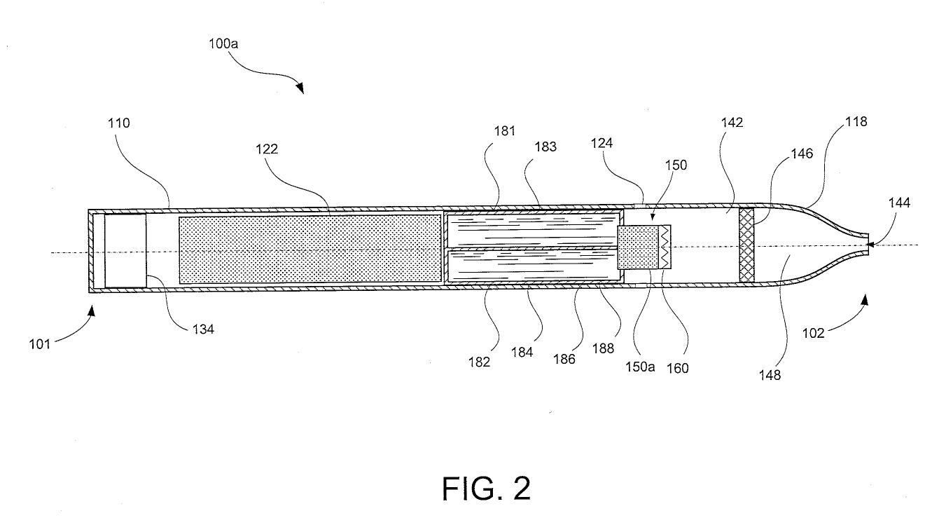

FIG. 2 illustrates a personal vaporizer 100a according to an embodiment of the invention that differs slightly from the vaporizer 100 of FIG. 1. As shown in FIG. 2, most of the components of the personal vaporizer 100a are the same as or similar to those of the personal vaporizer 100. In the vaporizer 100a, however, the fluid transport arrangement 150 comprises a single fluid transport structure 150a. The fluid transport structure 150a and the reservoirs 181, 182 are configured so that both fluids 183, 184 are transported from the reservoirs 181, 182 into the vaporization chamber by the single structure 150a. The fluid transport structure 150a may be or comprise a wick or collection of wicking material.

It will be understood that, as they are passed through the structure 150a, the two liquids 183, 184 will commingle and be mixed together to some degree. In some embodiments, the fluid transport structure 150a may be tailored to enhance or limit the degree of commingling of the fluids 183, 184 prior to their exposure to the heating element 160.

As with the embodiment of FIG. 1, the personal vaporizer 100a is not limited to two fluid reservoirs/vaporizable fluids. The reservoir section of the personal vaporizer 100a could, for example, be divided into three (or more) reservoirs instead of the two semi-cylindrical reservoirs 181, 182 of FIG. 1. In such an embodiment, each reservoir may contain a different vaporizable fluid which would all be transported to the vaporization chamber by the single transport structure 150a.

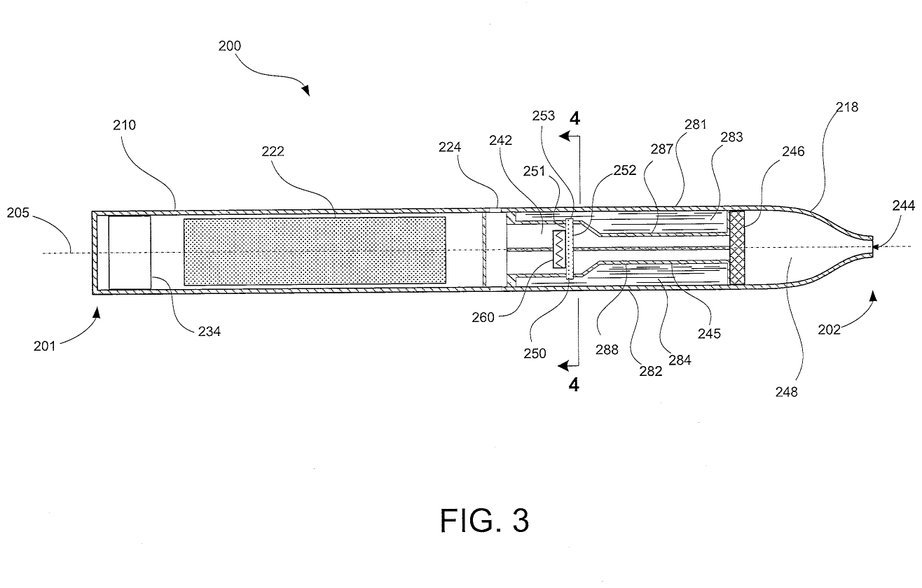

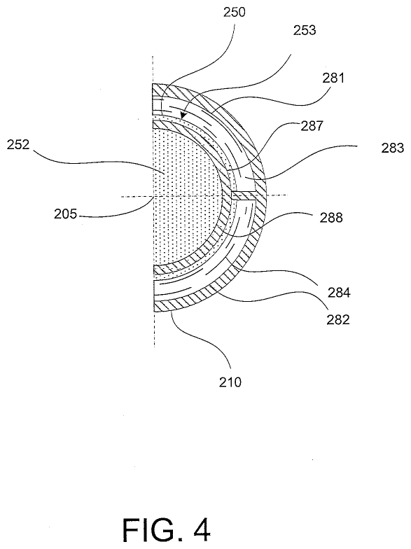

With reference to FIGS. 3 and 4, a personal vaporizer 200 according to another illustrative embodiment of the invention comprises a cylindrical casing 210 having a distal end 201 and a proximal end 202. As in the previous embodiments, the vaporizer 200 also has a mouthpiece 218 having an exit passage 244, a filter 246 and an exit chamber 248. The casing 210 also has one or more air holes 224 to allow air to flow from the atmosphere into a vaporization chamber 242. The personal vaporizer 200 also comprises a battery 222 and a control unit 234.

As in the previous embodiments, the personal vaporizer 200 includes two annular semicylindrical vaporizable fluid reservoirs 281, 282 configured for receiving first and second vaporizable fluids 283, 284, respectively. In this embodiment, however, the fluid reservoirs 281, 282 are positioned so that they collectively form a cylindrical wall surrounding some or all of the vaporization chamber 242 and the heating element 260. The heating element 260 is positioned at or near the proximal end of the vaporization chamber 242, which is in fluid communication with a chimney 245 bounded by the inner walls 287, 288 of the reservoirs 281, 282. The chimney 245 provides a conduit through which air and vaporization products pass from the vaporization chamber 242 to the filter 246 and exit chamber 248.

To supply fluid for vaporization by the heating element 260, the personal vaporizer 200 is provided with a disc-like fluid transport structure 250 having distal, proximal and circumferential surfaces 251, 252, 253. The fluid transport structure 250 is centered on the longitudinal axis 205 of the personal vaporizer 200 so that its distal surface 251 is adjacent or in contact with the heating element 260. The fluid transport structure 250 is sized so that it extends outward to and through arcuate openings in the inner walls of the fluid reservoirs 281, 283. The fluid transport structure 250 is configured so that fluids in the reservoir 280 are drawn through the circumferential surface 256 and/or through portions of the distal and proximal surfaces 251, 252 adjacent the circumferential surface 256. The fluid transport structure 250 is further configured so that the vaporizable liquids 283, 284 are both drawn inwardly toward the longitudinal axis of the personal vaporizer 200 and proximally toward the proximal surface 252 where they are exposed to heat from the heating element 260 and vaporized. The fluid transport structure 250 may be or comprise any of the previously described wick structures/materials. As in the vaporizer 100a, the use of a single fluid transport structure 250 will result in a degree of mixing of the two vaporizable fluids 283, 284 prior to vaporization.

It will be understood that the personal vaporizer 200 is not limited to two fluid reservoirs/vaporizable fluids. The reservoir section of the personal vaporizer 200 could, for example, be divided into three (or more) reservoirs instead of the two semi-cylindrical reservoirs 281, 282 of FIGS. 3 and 4. In such an embodiment, each reservoir may contain a different vaporizable fluid which would be transported to the vaporization chamber by the single transport structure 250.

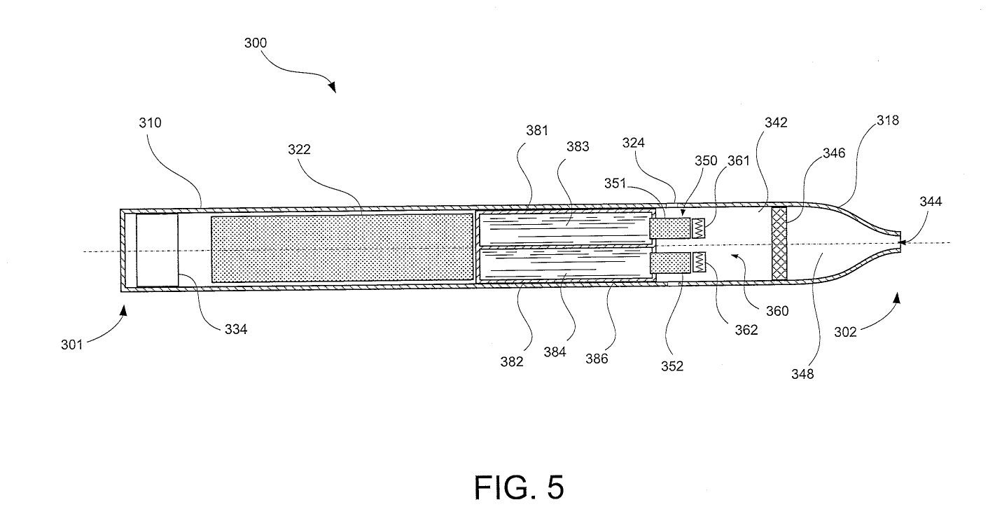

With reference to FIG. 5, a personal vaporizer 300 comprises a cylindrical casing 310 having a distal end 301 and a proximal end 302. As in the previous embodiments, the vaporizer 300 also has a mouthpiece 318 having an exit passage 344, a filter 346 and an exit chamber 348. The casing 310 also has one or more air holes 324 to allow air to flow from the atmosphere into a vaporization chamber 342. The personal vaporizer 300 also comprises a battery 322 and a control unit 334.

The personal vaporizer 300 further comprises two fluid reservoirs 381, 382 in which are disposed respective vaporizable fluids 383, 384. The fluid reservoirs 381, 382 may be configured as simple semicylinder tanks in which the fluids 383, 384 are disposed. In some embodiments, either or both of the reservoirs 381, 382 may be or include a housed or unhoused adsorptive or absorptive material or structure that retains the vaporizable fluid 383, 384. The vaporizer 300 also includes a fluid transport arrangement 350 that is configured for drawing the fluids 383, 384 into the vaporization chamber 342. The arrangement 350 includes a first fluid transport structure 351 configured and positioned to be in contact with the vaporizable fluid 383 in the first reservoir 381 and for drawing the fluid 383 out of the reservoir 381 into the vaporization chamber 342 and into close proximity or in contact with a first heating element 361 of a heating arrangement 360. The arrangement 350 also includes a second fluid transport structure 352 configured and positioned to be in contact with the vaporizable fluid 384 in the second reservoir 382 and for drawing the fluid 384 out of the reservoir 382 into the vaporization chamber 342 and into close proximity or in contact with a second heating element 362 of the heating arrangement 360. The heating elements 361, 362 may be configured to heat the vaporizable fluids 383, 384 through any conductive, convective, and/or radiative heat transfer mechanism. Either or both of the heating elements 361, 362 may be or include a resistance element in the form of a wire coil.

The fluid transport structures 351, 352 may be configured and positioned substantially similarly to those of the personal vaporizer 100 of FIG. 1. As in that embodiment, the structures 351, 352 may have different characteristics and/or flow properties tailored to their respective vaporizable fluids 383, 384. The fluid transport structures 351, 352 of the personal vaporizer 300 may each be or comprise a wick or collection of wicking material.

The primary difference between the personal vaporizer 300 of FIG. 4 and the personal vaporizer 100 of FIG. 1 is the use of two separate heating elements 361, 362. This configuration allows the use of different structure, heating characteristics, and position to be varied depending on the liquid to be vaporized by a particular heating element. The characteristics (e.g., temperature range) of the first heating element may be selected to provide optimum performance in vaporizing the first vaporizable liquid while the characteristics of the second heating element may be selected to provide optimum performance in vaporizing the second vaporizable liquid. The use of two heating elements can also provide the capability to separately activate the two elements. For example, in some applications, it may be desirable to initially activate only the heating element associated with one of the two fluids and then, after a delay activate the other heating element.

As in the previous embodiments, the personal vaporizer 300 is not limited to two fluid reservoirs/vaporizable fluids. The reservoir section of the personal vaporizer 100 could, for example, be divided into three (or more) reservoirs instead of the two semi-cylindrical reservoirs 381, 382 of FIG. 5. In such an embodiment, each reservoir may contain a different vaporizable fluid and the fluid transport arrangement 150 may include a fluid transport structure for each reservoir. There may also be a separate heating element adjacent the proximal end of each such fluid transport structure.

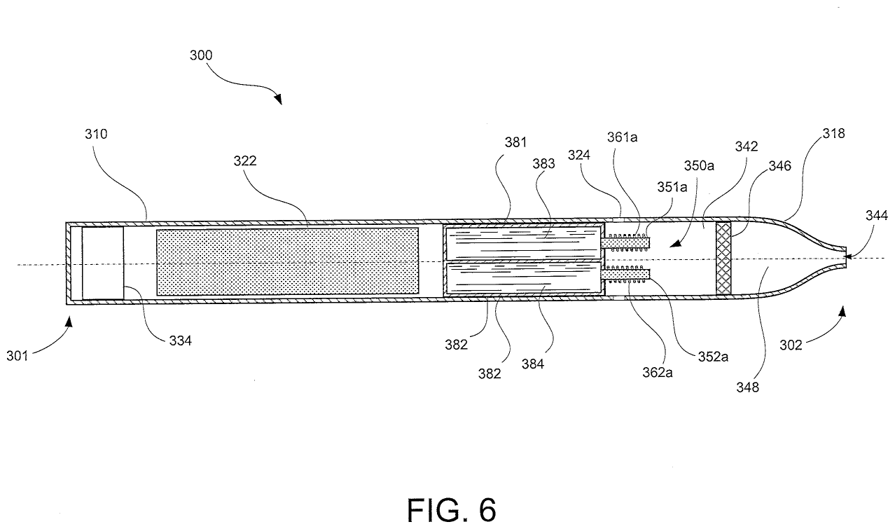

FIG. 6 illustrates a personal vaporizer 300a according to an embodiment of the invention that differs slightly from the vaporizer 300 of FIG. 4. As shown in FIG. 6, most of the components of the personal vaporizer 300a are the same as or similar to those of the personal vaporizer 300. In the vaporizer 300a, however, the heating elements 361a, 362a are each a coiled resistance heating element that surrounds at least a portion of a respective one of the fluid transport structures 351a, 352a. It will be understood that, in another similar embodiment, one of the two fluid transport structure/heating element combinations could be configured like those of FIG. 5 while the other could be configured like those of FIG. 6. It will also be understood that more than two reservoir/transport structure/heating combinations may be used.

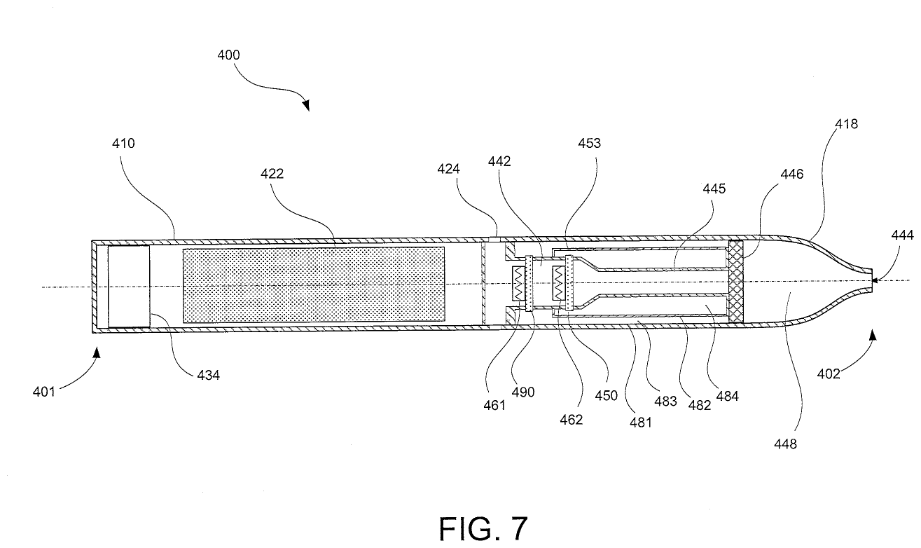

With reference to FIG. 7, a personal vaporizer 400 according to another illustrative embodiment of the invention comprises a cylindrical casing 410 having a distal end 401 and a proximal end 402. As in the previous embodiments, the vaporizer 400 also has a mouthpiece 418 having an exit passage 444, a filter 446 and an exit chamber 448. The casing 410 also has one or more air holes 424 to allow air to flow from the atmosphere into a vaporization chamber 442. The personal vaporizer 400 also comprises a battery 422 and a control unit 434.

The personal vaporizer 400 has two concentric axisymmetric vaporizable fluid reservoirs 481, 482 a portion of each of which surrounds a portion of the vaporization chamber 442. The first fluid reservoir 481 contains a first vaporizable fluid 483 and the second fluid reservoir 482 contains a second vaporizable fluid 484. The portion of the first reservoir 481 that surrounds a portion of the vaporization chamber 442 also surrounds a first heating element 461. The portion of the second reservoir 482 that surrounds a portion of the vaporization chamber 442 also surrounds a second heating element 461. The first heating element 461 is positioned within the vaporization chamber 442 distal to the second heating element 461, which is positioned at or near the proximal end of the vaporization chamber 442. The vaporization chamber 442 is in fluid communication with a chimney 445 bounded by the inner wall of the second fluid reservoir 482. The chimney 445 provides a conduit through which air and vaporization products pass from the vaporization chamber 442 to the filter 446 and exit chamber 448.

The personal vaporizer 400 is provided with a fluid transport arrangement having two disc-like fluid transport structures 450, 490 each having distal, proximal and circumferential surfaces. The first fluid transport structure 450 is centered on the longitudinal axis of the personal vaporizer 400 so that its distal surface is adjacent or in contact with the first heating element 461. The first fluid transport structure 450 is sized so that it extends outward to and through a circumferential opening in the inner wall of the first fluid reservoir 481. The second fluid transport structure 490 is centered on the longitudinal axis of the personal vaporizer 400 so that its distal surface is adjacent or in contact with the second heating element 462. The second fluid transport structure 490 is sized so that it extends outward to and through a circumferential opening in the inner wall of the second fluid reservoir 482.

The fluid transport structures 450, 490 are configured so that the fluids in the reservoirs 481, 482 are drawn through the circumferential surfaces of the structures 450, 490 and/or through portions of the distal and proximal surfaces adjacent the circumferential surface. The fluid transport structures 450,490 are further configured so that the vaporizable fluids 483, 484 are both drawn inwardly toward the longitudinal axis of the personal vaporizer 400 and proximally toward the proximal surfaces of the transport structures 450, 490 whereby the first vaporizable fluid 483 is exposed to the first heating element 461 and the second vaporizable fluid 484 is exposed to heat from the second heating element 462. The fluid transport structures 450, 490 may be or comprise any of the previously described wick structures/materials. As in the vaporizers 300 and 300a, two heating elements 461, 462 may have different structures and/or heating characteristics, and may be configured for separate activation.

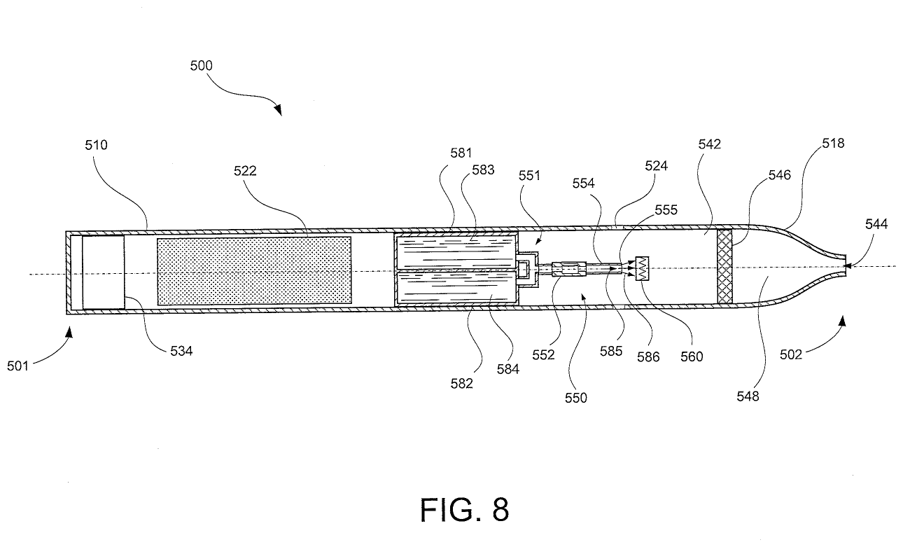

While some of the foregoing embodiments produce a degree of mixing of the fluids prior to vaporization, other embodiments of the invention may be configured specifically to produce thorough mixing of the fluids. With reference to FIG. 8, one exemplary personal vaporizer 500 of this type comprises a cylindrical casing 510 having a distal end 501 and a proximal end 502. As in the previous embodiments, the vaporizer 500 also has a mouthpiece 518 having an exit passage 544, a filter 546 and an exit chamber 548. The casing 510 also has one or more air holes 524 to allow air to flow from the atmosphere into a vaporization chamber 542. The personal vaporizer 500 also comprises a battery 522 and a control unit 534.

The personal vaporizer 500 further comprises two fluid reservoirs 581, 582 in which are disposed respective vaporizable fluids 583, 584. As in some of the previous embodiments, the fluid reservoirs 581, 582 may be configured as simple semicylinder tanks in which the fluids 583, 584 are disposed. The vaporizer 500 also includes a fluid transport arrangement 550 that is configured for drawing the fluids 583, 584 from their respective reservoirs 581, 582 and then mixing them before passing them into the vaporization chamber 542. In the illustrated embodiment, the fluid transport arrangement 550 includes a conduit arrangement 551 in communication with and extending proximally from the fluid reservoirs 581, 582. A piezoelectric (PZE) pump 552 connects the conduit arrangement 551 to a downstream conduit 554. The conduit arrangement 551 and the PZE pump 552 are configured for drawing the vaporizable fluids 583, 584 from the reservoirs 581, 582 combining and mixing the fluids and controlling the combined flow of fluid 587 through the downstream conduit 554.

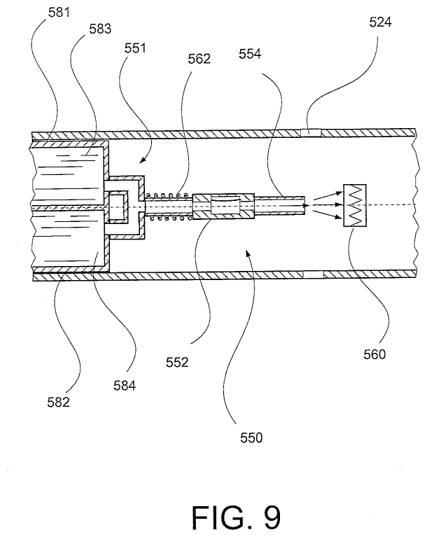

The fluid transport arrangement 550 may include a preheating arrangement configured for preheating the fluids 583, 584 upstream of the PZE pump 552 to facilitate flow through the PZE pump 552. One example of such an arrangement is shown in FIG. 9. In this example, a coiled heating element 562 is positioned to surround a portion of the conduit arrangement 551 upstream of the pump 552. In other embodiments, one or more heating elements could be positioned to preheat one or both of the fluids 583, 584 prior to their mixture. In some embodiments, heating elements could be positioned to heat one or both fluids within its respective reservoir or immediately adjacent the exit from the reservoir.

The downstream conduit 554 terminates in a nozzle end 555 that is positioned in close proximity or in contact with the heating element 560, which is configured to heat and vaporize the combined fluid 585. The resulting vapor products are mixed with air drawn in through the ports 524 before passing through to the exit chamber 548. As before, the heating element 560 may be configured to heat the vaporizable fluid 585 through any conductive, convective, and/or radiative heat transfer mechanism. The nozzle end 555 may be configured to direct the fluid 585 in a particular disbursement pattern to enhance vaporization performance.

Operation of the PZE pump 552 may be controlled through the use of a control module (not shown), which may be part of the control unit 534. The PZE pump 552 and the control module may be configured to control the rate of flow of the combined fluid 585 and, thus, the amount of fluid 585 delivered to the vaporization chamber 542. The PZE pump 552 may also be configured to selectively close off the conduit arrangement 551 to prevent unwanted flow of fluid from the reservoirs 541, 542.

It will be understood that the configuration and relative positioning of the components of the personal vaporizer 500 may be widely varying. In some embodiments, for example, the conduit arrangement 551 may comprise one or more flexible tubes.

Use of the personal vaporizer 500 is substantially similar to that of the previously described embodiments except that activation of the heating element 560 may also activate the pump PZE pump 552, causing fluids 583, 584 to be drawn from the reservoirs 581, 582, combined and passed into the vaporization chamber 530 where the combine fluid is heated to vaporization by the heating element 560. The resulting vapor mixes with air drawn through the air holes 524 and the mixture is drawn through the filter 546 and the exit chamber 548 and out through the mouthpiece exit passage 544. In some embodiments, an external switch or other control (not shown) in communication with the controller 534 may be manipulated by the user to change the flow rate of the combined fluid 585, which effectively changes the amount of fluid vaporized and the rate of delivery of active materials in the inhaled vaporization products.

In a variation of the above-described embodiment, the fluid transport arrangement 550 includes two PZE pumps, one for each fluid reservoir and fluid. In this embodiment, the ducts downstream of the PZE pumps may join to form a single duct in which the two fluids are combined or may each be configured with a nozzle directed so that the fluids mix just as they are introduced into the vaporization chamber. In yet another variation, the vaporizer could include two heating elements positioned within the vaporization chamber. In this embodiment, the downstream ducts from the two PZE pumps would each have a nozzle positioned to disperse one of the two fluids adjacent one of the two heating elements.

Similar embodiments to that shown in FIG. 8 can be constructed in which the passages are sized and configured so that the precursor liquids are drawn from their respective reservoirs by capillary action and/or vacuum/negative pressure gradients. In such embodiments, there may be no need for a pump to draw the liquids together for mixing and delivery to the vaporization chamber.

The liquids used in any of the above-described embodiments may be any miscible liquids. In particular embodiments, the first liquid may be a nicotine solution, while the second liquid may be a flavorant solution or a canabanoid solution. Maintaining and storing the liquids separately can allow easy variation in the relative concentrations in either or both solutions. In some embodiments, it can also allow variability in the relative concentration of the active material in one of the solutions relative to the active material in the other solution in the final mixture. The user could, for example, establish a use ratio of 0-100% of one liquid and 100-0% of the other. In multiple heating coil embodiments, this holds a significant advantage because the wick and coil materials/configuration may be vastly different for each liquid. For example, the best configuration for a nicotine solution may be a configuration with a cotton-based wick and a simple metal coil heating element while the best configuration for CBD and canabanoid solutions may involve the use of a predominantly ceramic or silica structure with very little traditional wicking material (if any). Using a two wick/two coil configuration allows one main device to perform at an optimum level with both solutions.

In embodiments using a single coil, the user may be able to control the relative concentrations in the mixed liquid prior to vaporization. For example, the user could increase or decrease the levels of each component depending on the time of day or as medicinal needs require.

The two liquids of the invention can also simply be two different flavorant solutions. This would allow the user to experiment with different flavorant combinations and different relative concentrations of two particular flavorants.

While the foregoing illustrates and describes exemplary embodiments of this invention, it is to be understood that the invention is not limited to the construction disclosed herein. The invention can be embodied in other specific forms without departing from the spirit or essential attributes. In particular, while the exemplary embodiments described above all employ two vaporizable fluids, the present invention is not limited to such embodiments and specifically encompasses other embodiments that provide for delivery of three or more vaporizable fluids.

1. A personal vaporizer comprising:
a vaporizer case defining a vaporizer interior;
a vaporization chamber within the vaporizer interior;
an air flow passage from one or more air intake openings in the case to the vaporization chamber;
a vaporization mixture flow passage extending from the vaporization chamber to an exit port;
a heating arrangement comprising at least one heating element disposed within the vaporization chamber, the heating arrangement being connected to a power source for selective activation by a user;
a first fluid reservoir configured for retaining a first vaporizable fluid within a first reservoir interior;
a second fluid reservoir configured for retaining a second vaporizable fluid within a second reservoir interior;
a fluid transport arrangement configured for transporting the first and second vaporizable fluids from the first and second fluid reservoirs into the vaporization chamber.

2. A personal vaporizer according to claim 1, wherein the fluid transport arrangement comprises:
a first fluid transport structure having a first portion in fluid communication with the first reservoir interior and a second portion disposed within the vaporization chamber, the first fluid transport structure being configured for transporting the first vaporizable fluid from the first fluid reservoir to the vaporization chamber; and
a second fluid transport structure having a first portion in fluid communication with the second reservoir interior and a second portion disposed within the vaporization chamber, the second fluid transport structure being configured for transporting the second vaporizable fluid from the second fluid reservoir to the vaporization chamber.

3. A personal vaporizer according to claim 2, wherein the first fluid transport structure comprises a first wick having a first set of wicking characteristics and the second fluid transport structure comprises a second wick having a second set of wicking characteristics that is different from the first set.

4. A personal vaporizer according to claim 2, wherein the heating arrangement comprises a first heating element disposed within the vaporization chamber adjacent the second portion of the first fluid transport structure and a second heating element disposed within the vaporization chamber adjacent the second portion of the second fluid transport structure.

5. A personal vaporizer according to claim 4, wherein the first and second heating elements each comprise a heating coil respectively surrounding the second portion of the first transport structure and the second portion of the second fluid transport structure.

6. A personal vaporizer according to claim 1, wherein the fluid transport arrangement comprises:
a fluid transport structure having a first portion in fluid communication with the first reservoir interior, a second portion in fluid communication with the second reservoir interior, and a third portion disposed within the vaporization chamber, the fluid transport structure being configured for transporting the first and second vaporizable fluids from the first and second fluid reservoirs, respectively, to the vaporization chamber.

7. A personal vaporizer according to claim 6,
wherein at least a portion of the first reservoir is a first annular semicylinder and at least a portion of the second reservoir is a second annular semicylinder, the first and second reservoirs being configured and positioned so that the first and second semicylinders are positioned in opposition so as to collectively define a cylindrical chamber wall surrounding the vaporization chamber, the chamber wall having a first opening between the vaporization chamber and the first reservoir and a second opening between the vaporization chamber and the second reservoir, and
wherein the first portion of the fluid transport structure extends through the first opening and the second portion of the fluid transport structure extends through the second opening.

8. A personal vaporizer according to claim 7 wherein the fluid transport structure comprises a disk-shaped wick having opposing planar surfaces, the first portion of the fluid transport structure being a first circumferential portion of the wick and the second portion of the fluid transport structure being a second circumferential portion of the wick and the wick being positioned so as to laterally span the vaporization chamber.

9. A personal vaporizer according to claim 8 wherein at least a portion of the at least one heating element is adjacent a central portion of one of the opposing planar surfaces of the disk-shaped wick.

10. A personal vaporizer according to claim 1, wherein the fluid transport arrangement comprises:
a conduit arrangement having an upstream portion in fluid communication with the first and second fluid reservoir interiors and a downstream portion in fluid communication with the vaporization chamber;
a pump operatively connected to the conduit arrangement so as to draw the first and second vaporizable fluids from the first and second fluid reservoir interiors through the upstream portion of the arrangement and to impel a mixture of the first and second vaporizable fluids into and through the downstream portion of the arrangement into the vaporization chamber.

11. A personal vaporizer according to claim 10, wherein the downstream portion of the conduit arrangement comprises a downstream conduit having a nozzle end extending into the vaporization chamber, the nozzle end being positioned adjacent the at least one heating element.

12. A personal vaporizer according to claim 10, wherein the nozzle end is configured to disburse the mixture of the first and second vaporizable fluids into the vaporization chamber in a predetermined pattern.

13. A personal vaporizer according to claim 10, wherein the pump is configured to controllably vary a flow rate of the mixture of the first and second vaporizable fluids into the vaporization chamber.

14. A personal vaporizer according to claim 10 wherein the fluid transport arrangement further comprises:
at least one preheating element configured and positioned to heat at least one of the first and second vaporizable fluids prior to mixture of the first and second vaporizable fluids.

15. A personal vaporizer according to claim 10 wherein the conduit arrangement is configured to produce the mixture of the first and second vaporizable fluids upstream of the pump and wherein the fluid transport arrangement further comprises:
at least one preheating element configured and positioned to heat the mixture of the first and second vaporizable fluids upstream of the pump.
